(19) United States
(12) Patent Application Publication
(10) Pub. No.: US 2024/0188930 A1
(43) Pub. Date: Jun. 13, 2024

(54) Adhesive Ultrasonic Sensing Device for Organ Detection

(71) Applicant: Decentralized Biotechnology Intelligence Co., Ltd., Taipei (TW)

(72) Inventors: Yao-Sheng Chou, Taipei (TW); Wei-Sheng Su, Taipei (TW); Hsiao-Yi Lin, Taipei (TW)

(21) Appl. No.: 18/470,640

(22) Filed: Sep. 20, 2023

(30) Foreign Application Priority Data
Dec. 8, 2022 (TW) 111147274

(51) Int. Cl. A61B 8/00; A61B 8/08

(52) U.S. Cl. CPC A61B 8/4236; A61B 8/0891; A61B 2562/164

(57) ABSTRACT

The present invention discloses an adhesive ultrasonic sensing device including a flexible substrate for attaching to a part of a body; a first electrode layer is configured on the flexible substrate; an ultrasonic sensor array is configured on the first electrode layer. A second electrode layer is disposed on the ultrasonic sensor array; and a thin film transistor layer, is configured on the second electrode layer. The ultrasonic sensor array emits ultrasonic waves to the part, and receives reflected ultrasonic waves from the part.

### TECHNICAL FIELD

The present invention relates to a sensor, more specifically, an adhesive ultrasonic sensing device for organ detection.

### BACKGROUND

Generally speaking, abnormal blood vessels are the precursors of diseases such as heart disease, cerebrovascular disease or hypertension, the aforementioned diseases all occupy a very high proportion of death causes. Therefore, if the blood vessels can be accurately treated when minor ailments occur, the risk of vascular abnormalities can be reduced.

Typically, the medical staffs will analyze the blood vessels of the patient by an ultrasonic sensing device when they would like to determine whether or not the patient's blood vessels are abnormal, However, there are tens of thousands of blood vessels in human body. Before operating the ultrasonic imaging device, the medical staff must operate the device to detect one by one among the numerous blood vessels if there is no simple way to find out the possible abnormal blood vessels. The detection process not only exhausts the doctors mentally, but also exhausts the patients by the lengthy analysis procedures. Namely, the traditional blood vessel detection method not only takes a long time, but also the device is too large to carry, the conventional detection way must be carried out by the medical staff, and it is impossible to monitor their own physical conditions by the patients whenever necessary.

In view of this, the present invention provides an improved sensing device with real-time monitoring capability to solve the poor efficiency caused by traditional sensing devices.

### SUMMARY OF THE INVENTION

Based on above, one aspect and purpose of the present invention is to provide an adhesive ultrasonic sensing device. The device includes a flexible substrate for attaching to a part of a body; a first electrode layer is configured on the flexible substrate; an ultrasonic sensor array is configured on the first electrode layer; a second electrode layer is disposed on the ultrasonic sensor array; and a thin film transistor layer, is configured on the second electrode layer; and wherein the ultrasonic sensor array emits ultrasonic waves to the part, and receives reflected ultrasonic waves for imaging.

The device further includes an ultrasonic controller to control ultrasonic signals generated by the ultrasonic sensor array. A photoelectric sensor is used to sense the part, the photoelectric sensor includes a light-emitting device and a photodetector. The detecting part includes, neck blood vessels, a heart, lungs, a liver, and pancreas.

In one aspect, the adhesive ultrasonic sensing device includes a first electrode layer; an ultrasonic emitting layer is configured on the first electrode layer; a first bias electrode layer is configured on the ultrasonic emitting layer; a thin film transistor layer, is configured on the first bias electrode layer; a pixel input electrode layer is configured on the thin film transistor layer; an ultrasonic receiving layer is configured on the pixel input electrode layer; a second bias electrode layer is configured on the ultrasonic receiving layer; a flexible substrate is disposed on the second bias electrode layer; and wherein flexible substrate is attached to a part of a body, the ultrasonic emitting layer emits ultrasonic waves to the part, and receiving reflected ultrasonic waves for inspection.

In one embodiment, the device further includes an ultrasonic controller to control ultrasonic signals generated by the ultrasonic emitting layer. The device further includes a photoelectric sensor to sense the part, the photoelectric sensor includes a light-emitting device and a photodetector.

In one embodiment, an integrated ultrasonic sensing device includes an ultrasonic sensing device over a flexible substrate; a piezoelectric sensor is over the flexible substrate; a diaphragm is configured on a circuit board; and a sound-insulating ring forms a resonant cavity with the circuit board, wherein the piezoelectric sensor is arranged under the sound insulating ring.

The integrated ultrasonic sensing device includes a first electrode layer; an ultrasonic emitting layer is configured on the first electrode layer; a first bias electrode layer is configured on the ultrasonic emitting layer; a thin film transistor layer, is configured on the first bias electrode layer; a pixel input electrode layer is configured on the thin film transistor layer; an ultrasonic receiving layer is configured on the pixel input electrode layer; a second bias electrode layer is configured on the ultrasonic receiving layer; a flexible substrate is disposed on the second bias electrode layer; and wherein flexible substrate is attached to a part of a body, the ultrasonic emitting layer emits ultrasonic waves to the part, and receiving reflected ultrasonic waves for imaging.

The integrated ultrasonic sensing device further includes an ultrasonic controller to control ultrasonic signals generated by the ultrasonic emitting layer and a photoelectric sensor to sense the part, the photoelectric sensor includes a light-emitting device and a photodetector.

### BRIEF DESCRIPTION OF THE DRAWINGS

FIG. 1 shows the adhesive ultrasonic sensing device of the present invention.

FIG. 2 shows the adhesive ultrasonic sensing device according to another embodiment of the present invention.

FIG. 3 shows the adhesive ultrasonic sensing device functional diaphragm of the present invention.

FIG. 4 shows the integrated type ultrasound sensing device for detecting heart sound according to one embodiment of the present invention.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

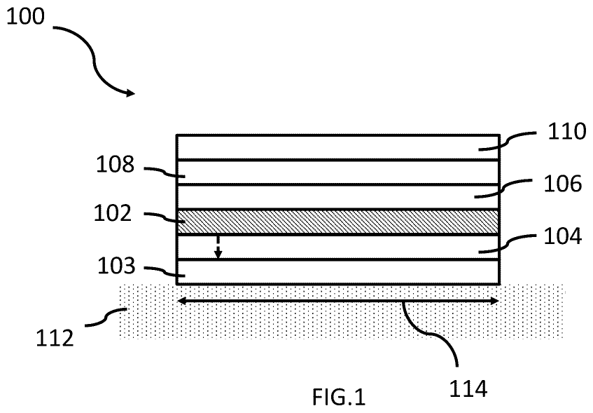

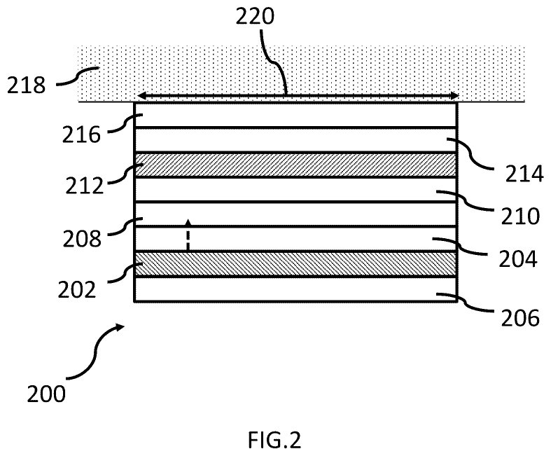

FIG. 1 shows a schematic cross-sectional view of a planar ultrasonic sensor of the adhesive sensing device according to an embodiment of the present invention. The imaging refers to capturing the image information of the internal organs, tissues, etc., within the body and perform imaging processing on an external device, thereby displaying the captured information on a monitor or display. The adhesive device is attached, covered, wrapped or worn on a certain body part under monitoring, such as the neck, chest (for example, monitoring the heart, liver, pancreas, lungs). The adhesive ultrasonic sensing device includes an anti-allergic gel layer formed at the bottom surface of the device. The anti-allergic gel layer contacts with the skin. The adhesive ultrasonic sensing device includes a planar ultrasonic sensor, please refer to FIG. 1 and FIG. 2. As shown in FIG. 1, the planar ultrasonic sensor 100 includes an ultrasonic sensor array 102, such as an ultrasonic transducer array 102. The first electrode layer 104 and the second electrode layer 106 are formed at both sides of the ultrasonic transducer array 102. The first electrode layer 104 is disposed on a flexible winding substrate 103. Pluralities of electrode units are formed on the first electrode layer 104. The second electrode layer 106 is disposed on the ultrasonic transducer array 102 and has electrode units corresponding to the first electrode layer 104. A thin film transistor (Thin Film Transistor) layer 108 is disposed on the second electrode layer 106. The thin film transistor layer 108 provides a substrate on which a pixel circuit array (pixels array) is formed, each pixel circuit includes at least one TFT element. The ultrasonic transducer array 102 is a piezoelectric layer formed of piezoelectric material which is selected from one of polyvinylidene fluoride (PVDF) and lead zirconate titanate piezoelectric ceramics (piezoelectric ceramic transducer, PZT) or the combination. The piezoelectric layer of the present invention includes a flexible piezoelectric film, such as a polyvinylidene fluoride thin film, so that the planar ultrasonic sensor 100 is flexible, it is beneficial for attaching body surface 112 under detecting. The planar ultrasonic sensor 100 is employed to construct a digital image by using the detected signals. The thin film transistor layer 108 includes a plurality of thin film transistors arranged in a matrix to form a thin film transistor array. For example, each TFT includes a gate formed on one side of the piezoelectric layer 102. The gate is covered by an insulating layer, a channel layer is formed on the insulating layer. A source and a drain layer are formed on the channel layer. In one embodiment, the thin film transistor is an organic thin film transistor (OTFT). The second electrode layer 106 is a bias electrode. The piezoelectric layer 102 also has the functions of sending and receiving ultrasonic sensing signals. Namely, the planar ultrasonic sensor 100 emits ultrasonic waves from the piezoelectric layer 102 and receives reflected ultrasonic waves, it refers to self-emitting/self-receiving architecture. For example, an ultrasonic controller (driver IC) is electrically connected with the first electrode layer 104 and the second electrode layer 106 to control the piezoelectric layer 102 for sending and receiving ultrasonic signals in different time periods. In one embodiment, the piezoelectric layer 102, the thin film transistor array 108, the first electrode layer 104, the second electrode layer 106, and the back layer 110 are all transparent. The structure allows the light transmittances of layers are all greater than 95%, thereby offering transparent planar ultrasonic sensor 10. The back layer 110 is provided to absorb reflected ultrasonic waves. In one embodiment, the materials of the first electrode layer 104 and the second electrode layer 106 is selected from indium tin oxide (ITO), zinc oxide (ZnO), Poly(3,4-ethylenedioxythiophene)-polystyrenesulfonic acid (Poly(3,4-ethylenedioxythiophene), PEDOT), carbon nanotube (CNT), nano silver wire (Ag nano wire, ANW) and graphene.

As shown in FIG. 2, it shows a planar ultrasonic sensor 200 of another embodiment which includes an ultrasonic emitter and an ultrasonic receiver under a soft flexible substrate (adhesive layer) 216. The ultrasonic emitter is an ultrasonic wave generator comprising a substantially planar piezoelectric emitting layer 202. A first electrode layer 204 and a second electrode layer 206 are disposed on two sides of the piezoelectric emitting layer 202. The ultrasonic waves can be generated by applying a voltage to the piezoelectric emitting layer 202 via the first electrode layer 204 and the second electrode layer 206 for generating the ultrasonic waves. A plurality of electrode units is disposed on the first electrode layer 204. The second electrode layer 206 is a bias electrode layer disposed on the piezoelectric emitting layer 202 and has electrode units corresponding to the first electrode layer 204. The piezoelectric emitting layer 202 is formed with the piezoelectric material. The ultrasonic wave travels toward the site to be detected (adhesive site) 218 and passes through the substrate 216. The ultrasonic waves not absorbed by the site 218 is reflected back through the substrate 216 and received by the ultrasonic receiver. The first electrode layer 204 and the second electrode layer 206 may be metallized electrodes, such as metal layers coating both sides of the piezoelectric emitting layer 202.

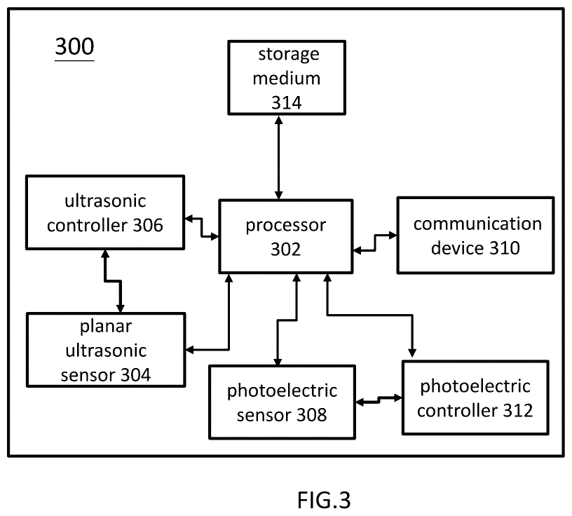

The ultrasonic receiver may include an array of pixel circuits formed on a thin film transistor layer 208, and a piezoelectric receiving layer 212. In some implementations, the TFT layer 208 includes a substrate containing an array of pixel circuits, each pixel circuit includes at least one TFT device, or additional circuit elements such as diodes, capacitors, and the like. Each pixel circuit can convert charges generated in the piezoelectric receiving layer 212 into electrical signals. Each pixel circuit may include a pixel input electrode electrically coupling the piezoelectric receiving layer 212 to the pixel circuit. The pixel input electrode layer 210 is disposed on the TFT layer 208. The ultrasonic receiving layer 212 is disposed on the pixel input electrode layer 210. A bias electrode 214 of the receiver is disposed on the piezoelectric receiving layer 212. The flexible substrate 216 is disposed on the bias electrode 214. The bias electrode 214 is a metallized electrode and is grounded or biased to control the signals to the TFT array. Ultrasonic energy reflected from the top surface of the flexible substrate 216 is converted into a local charge by the piezoelectric receiving layer 212, followed by collecting via the pixel input electrode 210 for delivering to the underlying pixel circuit. These charges are amplified by the pixel circuit and provided to the ultrasonic controller (refer to the FIG. 3) for subsequent imaging process.

The ultrasonic controller is coupled to the ultrasonic emitter and the ultrasonic receiver to generate one or more ultrasonic signals. The ultrasonic controller is electrically connected with the first electrode layer 204 and the second electrode layer 206, and is electrically connected with the bias electrode 214 and the pixel circuit on the thin film transistor layer 208 to control the piezoelectric emitting layer 202, receiving layer 212 transmits for emitting, receiving ultrasonic sensing signals. The substrate 216 includes a flexible material. The thickness of the piezoelectric emitting layer 202, receiving layer 212 is selected to produce and reception of ultrasound waves.

The FIG. 3 shows a functional block diagram of the adhesive imaging device according to another embodiment of the present invention. In this embodiment, the adhesive imaging device 300 includes: a processor 302, a planar ultrasonic sensor 304, an ultrasonic controller 306, a photoelectric sensor 308, a communication device 310, a photoelectric controller 312 and storage medium 314. The processor 302 is coupled to the planar ultrasonic sensor 304, the ultrasonic controller 306, the photoelectric sensor 308, the communication component 310, the photoelectric controller 312 and the storage medium 314 for controlling these components.

The photoelectric sensor 308 is used for sensing a blood vessel or organ to be measured (for instant, heart, lung, liver, pancreas, etc.) for generating a sensing signal. In one embodiment, the photoelectric sensor 308 includes a photoelectric substrate, a light-emitting device, and a photodetector. The surface of the photoelectric substrate is provided with an emitting area and a receiving area, wherein the light-emitting element is arranged in the emitting area to emit light to the blood vessel or organ under measuring. The light detector is arranged in the receiving area to receive the light reflected from the blood vessel or organ, thereby generating the sensing signal according to the reflected light.

In one embodiment, the light-emitting device includes a micro-light-emitting diode array (or mini LED array), it emits light of several different wavelengths (red light, green light, and blue light), The light of several different wavelengths is emitted toward the subject's blood vessel or organ under measuring, even if some light is not capable to pass through human tissues, however, some other light may still penetrates human tissues to the blood vessel or organ due to different light characteristics, thereby generating desired sensing signals. With the arrangement of the micro-light-emitting diode array, the light-emitting device has the characteristics of lower energy consumption, smaller volume size, and more fine light to irradiate to the measuring parts, accurately.

The photoelectric controller 312 is electrically connected to the photoelectric sensor 308 for controlling several light emitting sections to sequentially emit light of different wavelengths along the scanning direction. In other words, the photoelectric controller 312 controls the array of micro light emitting diodes to emit light sequentially, randomly or globally. Accordingly, when several light-emitting sections emit light of different wavelengths toward the blood vessel or organ to be measured, the light detector can completely receive the light reflected from the blood vessel or organ, so that the light detector can properly receive the signals, the present invention improves the sensing accuracy.

The processor 302 is electrically connected to the photoelectric sensor 308 to receive the sensing signal, and converts the sensing signal into a blood vessel flow state data. When the blood vessel flow state data is within a reference flow state range, the processor 302 outputs a driving signal. The processor 302 determines the light absorption of the blood according to the sensing signal, and calculates the blood flow rate, the blood vessel size, or the blood oxygen level based on the data. The blood vessel flow state data includes blood flow rate value and blood oxygen content, and the reference flow state range which sets a reference flow rate range and a reference blood oxygen level range to determine whether or not the vessel is abnormal. The processor 302 can simply find out the possible occurrence of blood vessel abnormalities according to the sensing results generated by the photoelectric sensor 308, and output a driving signal when the blood vessel is abnormal. The planar ultrasonic sensor 304 can quickly detect the place where the blood vessel abnormality may occur, thereby improving the detection efficiency.

The ultrasonic controller 306 receives reflected ultrasonic signals from the ultrasonic receiver. The ultrasonic controller 306 employs the signal received by the ultrasonic receiver to construct a digital image of the area 220 under detecting 218 through the processor 302. In some embodiments, the ultrasound controller 306 may also sample the output signal over time to detect blood flow in the neck vessels or the images of other body organs. The planar ultrasonic sensor 304 is electrically connected to the processor 302, so that the processor 302 generates blood vessel diameter data after receiving the driving signal. The planar ultrasonic sensor 304 is provided, for example, illustrated in FIGS. 1 and 2, to calculate the caliber of the blood vessel while performing blood vessel imaging, when the blood vessel caliber is larger or smaller than the predetermined data, different warning messages are output based on the detected caliber. According to embodiment, the present invention can simply find out the possible occurrence of blood vessel abnormality through the photoelectric sensor 308, the planar ultrasonic sensor 304 is driven by the processor 302 to further detect blood vessel abnormality. In addition to real-time monitoring, the present invention also improves the detection efficiency. In another example, the photoelectric sensor 308 and the planar ultrasonic sensor 304 can also detect images of other organs of the body, such as the heart, lungs, liver, pancreas, etc., for disease diagnosis and screening. Ultrasonic image processing can be achieved by the processor and computing technology. Therefore, redundancy description is omitted here.

A display receives and displays the blood vessel flow state data and the blood vessel caliber data from processor 302, for the medical staff through the display to check the sensing results detected by the photoelectric sensor 308 and the planar ultrasonic sensor 304.

According to another embodiment, the ultrasonic imaging method of the present invention includes the following steps: employing the ultrasonic controller 306 to control the pulse repetition interval (pulse repetition interval, PRI) to transmit a plurality of ultrasonic signals to the site to be detected (adhesive site); subsequently, the planar ultrasonic sensor 304 receives a plurality of reflected signals; The artificial intelligence (AI) or a neural network separates the reflected signal into blood flow signals and clutter signals. The processor calculates the blood flow parameters according to the blood flow signals, and determine the blood vessel position according to the blood flow parameter. Finally, The processor adjusts the image signals corresponding to the reflected signal according to the blood flow parameter and the blood vessel position, so as to generate the ultrasound image. The above calculations are performed by the processor 302. The neural network may be a Convolution Neural Network (CNN) or other similar neural networks. The neural network has been pre-trained to separate the reflected signal of the ultrasound signal into the blood flow signal and the clutter signal. In one example, multiple sets of training samples may be prepared in advance, wherein each set of training samples includes multiple sets of reflected signals of ultrasonic signals, and blood flow signals and clutter signals separated from the reflected signals of the ultrasonic signals. The training samples are input into the neural network to train the neural network to separate the reflected signal of the ultrasonic signal into the blood flow signal and the clutter signal.

The driving circuit of the planar ultrasonic sensor 304 can transmit or receive ultrasonic waves by active or passive driving methods. As an example, the image display of the light-emitting array is driven and controlled by an image driver IC, and the image driver IC is electrically connected to the control circuit board through a flexible connection circuit.

In one embodiment, the user of the adhesive imaging device 300 can communicate with another device (such as a smart phone, a tablet computer) through the communication device 310, for example, by a wireless network including bluetooth, WLAN, Wifi, 3G, 4G, 5G and other wireless networks of various wireless specifications.

In one embodiment, the storage medium 314 can store image data or application software. The storage medium 314 includes, for example, a memory and a computer-readable medium. The memory stores software or programs that can be run by the processor. Memory includes non-permanent memory, random access memory (RAM) and/or non-volatile memory, such as read only memory (ROM) or flash memory (Flash RAM). Storage media include, but are not limited to: phase change memory (PRAM), static random access memory (SRAM), dynamic random access memory (DRAM), other types of random access memory (RAM) or read-only memory to store information that can be accessed by a computing device.

In one embodiment, the adhesive imaging device is configured to communicate with an external device, which may be an external computing device, computing system, mobile device (smart phone, tablet computer, smart watch . . . etc.), or other electronic device types.

External devices include computing cores, user interfaces, internet interfaces, wireless communication transceivers, and storage devices. The user interface includes one or more input devices (for example, keyboard, touch screen, voice input device, etc.), one or more audio output devices (for example, speakers, etc.) and/or one or more visual output devices (for example, video graphics displays, touch screens, etc.). The internet interface includes one or more network devices (for example, wireless local area network (WLAN) devices, wired LAN devices, wireless wide area network (WWAN) devices, etc.). Storage devices include flash memory devices, one or more hard disk drives, one or more solid state storage devices and/or cloud storage.

A computing core includes a processor and other computing core components. Other computing core components include a video graphics processing unit, a memory controller, main memory (RAM), one or more input/output (I/O) device interface modules, input/output (I/O) interfaces, input/output (I/O) controller, peripheral device interface, one or more USB interface modules, one or more network interface modules, one or more memory interface modules and/or one or more Peripheral device interface module. The external device processes the data transmitted by the communication device 310 to generate various results.

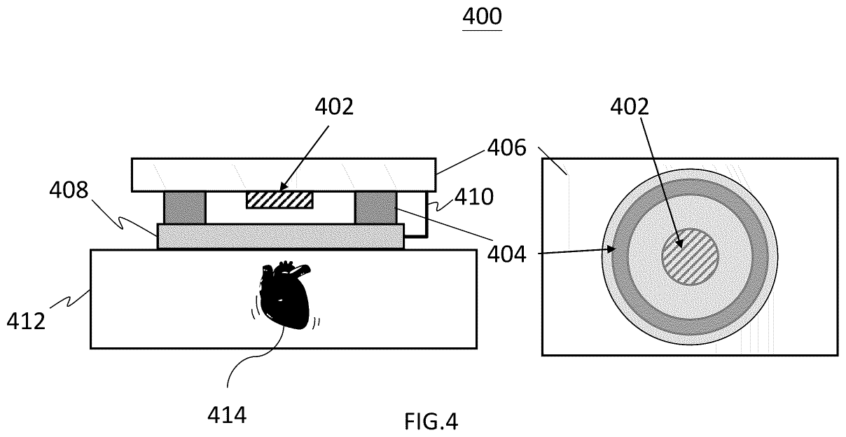

The FIG. 4 shows an integrated heart sound ultrasonic sensing device according to another embodiment of the present invention, it integrates the aforementioned planar ultrasonic sensor into a stethoscope. In practice, the user may perform the heart sound measurement first, and subsequently, the cardiac ultrasound examination is processed, directly, when the heart sound is abnormal. The integrated heart sound ultrasonic sensing device 400 includes a diaphragm 402 plated with conductive material, and the diaphragm 402 is packaged with a plastic frame (sound insulation ring) 404 and a circuit board 406 (the sound insulation ring 404 and the circuit board 406 form a resonant cavity. A microphone structure is created by the combination of the resonant cavity with the diaphragm 402). A piezoelectric sensor 408 is arranged below the sound insulating ring 404, and is electrically connected to the circuit board 406 through a line 410. The voltage signal generated by the vibration is measured by contacting with the human skin 412. As known in the art, the traditional capacitive sensor is poor to detect the frequency around 20 Hz. In this embodiment, the piezoelectric sensor 408 integrates the vibrating membrane to assist not only the traditional capacitive sensors in detecting low-frequency signals, but also performs the ultrasonic sensing for both heart sound sensing and ultrasonic sensing. The structure of the piezoelectric sensor 408 is that the ultrasonic emitter and the heart sound receiver arranged in the piezoelectric emitting layer 202 in FIG. 2, so that the integrated heart sound ultrasonic sensing device 400 has heart sound capture and ultrasonic detection dual functions.

In one embodiment, the piezoelectric sensor 408 is formed on a flexible substrate with a patch form. Pluralities of electrodes are arranged at the bottom of the flexible substrate to determine whether the patch attachment is set or not. In one embodiment, the patch can be directly pasted on the skin 412 above the user's heart 414 for measuring heart sound signals.

The above-mentioned sensing device is employed mainly to receive a signal including not limited to heartbeat signal. The integrated heart sound ultrasonic sensing device disclosed by the present invention can receive the heart beating signal and the ultrasonic signal by the capacitive sensor and the piezoelectric sensor, respectively. In the example, the circuit board includes amplifiers, filters, power, a management system, an identification system, bluetooth and processor, as well as the components included in FIG. 3.

As will be understood by persons skilled in the art, the foregoing preferred embodiment of the present invention illustrates the present invention rather than limiting the present invention. Having described the invention in connection with a preferred embodiment, modifications will be suggested to those skilled in the art. Thus, the invention is not to be limited to this embodiment, but rather the invention is intended to cover various modifications and similar arrangements included within the spirit and scope of the appended claims, the scope of which should be accorded the broadest interpretation, thereby encompassing all such modifications and similar structures. While the preferred embodiment of the invention has been illustrated and described, it will be appreciated that various changes can be made without departing from the spirit and scope of the invention.

What is claimed is:

1. An adhesive ultrasonic sensing device, comprising:
a flexible substrate for attaching to a part of a body;
a first electrode layer configured on said flexible substrate;
an ultrasonic sensor array configured on said first electrode layer;
a second electrode layer disposed on said ultrasonic sensor array; and
a thin film transistor layer, configured on said second electrode layer, including a pixel array; and
wherein said ultrasonic sensor array emits ultrasonic waves to said part, and receives reflected ultrasonic waves for imaging.

2. The device of claim 1, further including an ultrasonic controller to control ultrasonic signals generated by said ultrasonic sensor array.

3. The device of claim 1, further including a photoelectric sensor to sense said part, said photoelectric sensor including a light-emitting device.

4. The device of claim 3, further including a photoelectric sensor to sense said part, said photoelectric sensor including a photodetector.

5. The device of claim 1, wherein said part includes, neck blood vessels, a heart, lungs, a liver, and pancreas.

6. An adhesive ultrasonic sensing device, comprising:
a first electrode layer;
an ultrasonic emitting layer configured on said first electrode layer;
a first bias electrode layer configured on said ultrasonic emitting layer;
a thin film transistor layer, configured on said first bias electrode layer, including a pixel array;
a pixel input electrode layer configured on said thin film transistor layer;
an ultrasonic receiving layer configured on said pixel input electrode layer;
a second bias electrode layer configured on the ultrasonic receiving layer;
a flexible substrate disposed on said second bias electrode layer; and
wherein flexible substrate is attached to a part of a body, said ultrasonic emitting layer emits ultrasonic waves to said part, and receiving reflected ultrasonic waves for imaging.

7. The device of claim 6, further including an ultrasonic controller to control ultrasonic signals generated by said ultrasonic emitting layer.

8. The device of claim 6, further including a photoelectric sensor to sense said part, said photoelectric sensor including a light-emitting device.

9. The device of claim 8, further including a photoelectric sensor to sense said part, said photoelectric sensor including a photodetector.

10. The device of claim 6, wherein said part includes, neck blood vessels, a heart, lungs, a liver, and pancreas.

11. An integrated ultrasonic sensing device, comprising:
an ultrasonic sensing device over a flexible substrate;
a piezoelectric sensor over said flexible substrate;
a diaphragm configured on a circuit board; and
a sound-insulating ring forms a resonant cavity with said circuit board, wherein said piezoelectric sensor is arranged under said sound insulating ring.

12. The device of claim 11, wherein said ultrasonic sensing device including:
a first electrode layer;
an ultrasonic emitting layer configured on said first electrode layer;
a first bias electrode layer configured on said ultrasonic emitting layer;
a thin film transistor layer, configured on said first bias electrode layer, including a pixel array;
a pixel input electrode layer configured on said thin film transistor layer;
an ultrasonic receiving layer configured on said pixel input electrode layer;
a second bias electrode layer configured on the ultrasonic receiving layer;
a flexible substrate disposed on said second bias electrode layer; and
wherein flexible substrate is attached to a part of a body, said ultrasonic emitting layer emits ultrasonic waves to said part, and receiving reflected ultrasonic waves for imaging.

13. The device of claim 12, further including an ultrasonic controller to control ultrasonic signals generated by said ultrasonic emitting layer.

14. The device of claim 12, further including a photoelectric sensor to sense said part, said photoelectric sensor including a light-emitting device.

15. The device of claim 14, further including a photoelectric sensor to sense said part, said photoelectric sensor including a photodetector.

16. The device of claim 11, wherein said part includes, neck blood vessels, a heart, lungs, a liver, and pancreas.
